(19) United States
(12) Patent Application Publication
(10) Pub. No.: US 2020/0171026 A1
(43) Pub. Date: Jun. 4, 2020

(54) PROSTACYCLIN RECEPTOR AGONISTS FOR REDUCTION OF BODY FAT

(71) Applicant: Allergan, Inc., Irvine, CA (US)

(72) Inventors: Yong-Xin Li, Irvine, CA (US); Neil J. Poloso, Lake Forest, CA (US); John E. Donello, Dana Point, CA (US); Qianyong Liu, Tustin, CA (US)

(21) Appl. No.: 16/634,532

(22) PCT Filed: Jul. 27, 2018
(86) PCT No.: PCT/US2018/044134

§ 371 Date: Jan. 27, 2020

Related U.S. Application Data
(60) Provisional application No. 62/537,853, filed on Jul. 27, 2017.

(51) Int. Cl. A61K 31/4965; A61K 31/27; A61K 31/343; A61K 31/191; A61P 3/04

(52) U.S. Cl. CPC A61K 31/4965; A61K 31/27; A61K 9/0014; A61K 31/191; A61P 3/04; A61K 31/343

(57) ABSTRACT

Prostacyclin (PGI2) analogues which are agonists of the prostacyclin receptor (PI) are demonstrated to activate lipolytic activity in adipocytes. Also described are pharmaceutical compositions and methods for using the PGI2 receptor agonists to reduce subcutaneous adipose tissue and to treat or reduce symptoms of obesity-related diseases or disorders such as diabetes mellitus, fatty liver disease and cardiovascular disease.

### CROSS-REFERENCE TO RELATED APPLICATIONS

This application is an international application which claims priority to and/or the benefit of U.S. provisional application 62/537,853, filed Jul. 27, 2017, which is hereby incorporated by reference herein in its entirety.

### TECHNICAL FIELD

The subject matter described herein relates to prostacyclin receptor agonists for reduction of body fat, and in particular to PGI₂ receptor agonists and analogs thereof which activate lipolysis in adipocytes. The agonists are useful in activating lipolysis in adipocytes, and in particular, in reducing local deposits of excess adipose tissue including cellulite and in treating or reducing symptoms of obesity-related disorders.

### BACKGROUND

Adipose tissue is the primary energy storage tissue of the body. Fat cells, or adipocytes, store this energy in the form of triglycerides. Triglycerides (TGs) are mobilized from fat stores to provide caloric energy to the body through triglyceride hydrolysis. This process releases free or non-esterified fatty acids (NEFA) and glycerol into the blood for use by other body tissues. The breakdown of triglycerides from fat stores is referred to as lipolysis. Strategies that aim to increase lipolysis would be useful in treating local deposits of excess adipose tissue and also cellulite where engorged adipocytes bulge and created an uneven surface appearance.

Excess fat in an individual can be undesirable for a number of reasons. In some cases, the excess fat can be aesthetically unpleasing, such as in the case of cellulite, and other externally visible fat deposits such as, for example, fat deposits in the submental (under the chin), abdominal, waist, and thigh regions. In other cases, excess fat can result in obesity, which can be associated with, and increase the likelihood of, a myriad of diseases and conditions such as, for example type 2 diabetes, sleep apnea, heart disease, some types of cancer, osteoarthritis, and others.

Many methods for fat reduction generally involve exercise and diet control. However, methods of fat reduction by administration of fat-reducing compounds offer advantages such as simplicity, ease of implementation, and an ability to target fat deposits in both systemic (e.g. throughout the whole body of the individual) and localized (e.g. directly to submental fat and/or cellulite deposits) manners. Consequently, there is a need for cosmetic fat reduction and therapeutic fat reduction.

### BRIEF SUMMARY

In one aspect, a method for increasing lipolysis in adipocytes is provided comprising exposing the adipocyte to a prostacyclin receptor agonist or a pharmaceutically acceptable salt thereof. In some embodiments, the method increases glycerol production by the adipocytes by at least about 50%, 75%, 100%, 150%, 200%, 250%, 300%, 350% or 400% or by about 50% to 400%, 50% to 300%, 50% to 200% or 100% to 400% as compared to glycerol production by adipocytes not treated with the agonist.

In another aspect, a method for reducing fat in a subject in need thereof is provided, comprising administering to the subject a prostacyclin (PGI₂) receptor agonist or a pharmaceutically acceptable salt thereof.

In some embodiments, the PGI₂ receptor agonist is selected from the group consisting of beraprost, iloprost, carbacyclin, cicaprost, treprostinil, FK-788, selexipag, or a pharmaceutically acceptable salt thereof. In some embodiments, the PGI₂ receptor agonist is an analogue of beraprost, iloprost, carbacyclin, cicaprost, treprostinil, FK-788, or selexipag. In still other embodiments, the PGI₂ receptor agonist is a prodrug of beraprost, iloprost, carbacyclin, cicaprost, treprostinil, FK-788, or selexipag.

In some embodiments, the reducing fat in the subject comprises reducing the size of adipocytes in the subject. In other embodiments, the adipocytes are located in subcutaneous adipose tissue of the subject. In still other embodiments, the adipocytes are not located in visceral adipose tissue.

In some embodiments, the administering is to a localized region of the subject. In still other embodiments, the localized region is selected from the group consisting of the buttocks, under the chin, arms, underarms, neck, face, under one or both eyes, cheek, brow, calf, back, hips, legs, thighs, knees, ankles, abdomen, stomach and combinations thereof.

In some embodiment, the administering is via a subcutaneous injection. In other embodiments, the administering is via an intradermal or transdermal injection. In still other embodiments, the administering is via a dermal patch or a subdermal depot. In yet other embodiments, the administering is topical.

In some embodiments, the subject is diagnosed with or at risk of obesity, diabetes mellitus, fatty liver disease or a cardiovascular disease or disorder. In other embodiments, the cardiovascular disease or disorder is hypertension or pulmonary artery hypertension.

In some embodiments, the method decreases insulin resistance.

In some embodiments, the method comprising administering the PGI₂ receptor agonist to the subject every other day, twice a week, once a week, once every 2 weeks, once every month, once every 2 months, once every 3 months, once every 4 months, once every 5 months, or once every 6 months.

Some example embodiments are listed below.

Example Embodiment 1: A method for reducing body fat in a subject in need thereof comprising administering to the subject in need thereof a pharmaceutical composition comprising a PGI2 receptor agonist.

Example Embodiment 2: The method of example embodiment 1, wherein the agonist is selected from the group consisting of selexipag, FK-788, beraprost, iloprost, carbacyclin, cicaprost and treprostinil, or a pharmaceutically acceptable salt thereof.

Example Embodiment 3: The method of example embodiment 1 or 2, wherein the administering is via a subcutaneous or a transdermal injection.

Example Embodiment 4: The method of example embodiment 1 or 2, wherein the administering is via a dermal patch, a transdermal patch, or a subdermal depot.

Example Embodiment 5: The method of example embodiment 1 or 2, wherein the administering is topical.

Example Embodiment 6: The method of any one of example embodiments 1 to 5, wherein the pharmaceutical composition is a sustained release formulation.

Example Embodiment 7: The method of any one of example embodiments 1 to 5, wherein the pharmaceutical composition is an immediate release formulation.

Example Embodiment 8: The method of any one of example embodiments 1 to 7, wherein the administering to the subject comprises administering the agonist to a region of the body selected from the group consisting of buttocks, under the chin, underarm, under one or both eyes, cheek, brow, calf, back, thigh, ankle, and abdomen.

Example Embodiment 9: The method of any one of example embodiments 1 to 8, wherein the subject has cellulite.

Example Embodiment 10: The method of example embodiment 9, wherein the administering is to an area within or near the cellulite.

Example Embodiment 11: The method of any one of example embodiments 1 to 10, wherein the subject is not or has not been diagnosed with pulmonary arterial hypertension.

Example Embodiment 12: The method of any one of example embodiments 1 to 10, wherein the subject is a human.

Example Embodiment 13: A method for treating a subject in need thereof comprising administering a pharmaceutical composition comprising PGI2 receptor agonist.

Example Embodiment 14: The method of example embodiment 13, wherein the subject is diagnosed with or at risk of obesity, diabetes mellitus, fatty liver disease, or a cardiovascular disease.

Example Embodiment 15: The method of example embodiment 12 or 13, wherein the agonist is selected from the group consisting of selexipag, FK-788, beraprost, iloprost, carbacyclin, cicaprost and treprostinil, or a pharmaceutically acceptable salt thereof.

Example Embodiment 16: The method of any one of example embodiments 1 to 13, wherein the subject is not otherwise in need of medication to treat obesity, diabetes mellitus, fatty liver disease, or a cardiovascular disease.

Example Embodiment 17: The method of any one of example embodiments 13 to 16, wherein the subject is a human.

Example Embodiment 18: A method for activating lipolysis in an adipocyte, comprising exposing the adipocyte to a PGI2 receptor agonist.

Example Embodiment 19: The method of example embodiment 18, wherein the activating lipolysis results in a 50% to 300% increase in the production of glycerol by the adipocyte.

Example Embodiment 20: The method of example embodiment 18 or 19, wherein the agonist is selected from the group consisting of selexipag, FK-788, beraprost, iloprost, carbacyclin, cicaprost and treprostinil, or a pharmaceutically acceptable salt thereof.

Example Embodiment 21: The method of any one of example embodiments 18 to 20, wherein the adipocyte is a human adipocyte.

Example Embodiment 22: Use of a PGI2 receptor agonist in the manufacture of a medicament for reducing body fat in a subject in need thereof.

Example Embodiment 23: The use of example embodiment 22, wherein the agonist is selected from the group consisting of selexipag, FK-788, beraprost, iloprost, carbacyclin, cicaprost and treprostinil, or a pharmaceutically acceptable salt thereof.

Example Embodiment 24: The use of example embodiment 22 or 23, wherein the medicament, when used in the reduction of body fat in a subject in need thereof, is administered via a subcutaneous or a transdermal injection.

Example Embodiment 25: The use of example embodiment 22 or 23, wherein the medicament, when used in the reduction of body fat in a subject in need thereof, is administered via a dermal patch, a transdermal patch, or a subdermal depot.

Example Embodiment 26: The use of example embodiment 22 or 23, wherein the medicament, when used in the reduction of body fat, is administered topically.

Example Embodiment 27: The use of any one of example embodiments 22 to 26, wherein the medicament is a pharmaceutical composition which is a sustained release formulation.

Example Embodiment 28: The use of any one of example embodiments 22 to 26, wherein the medicament is a pharmaceutical composition which is an immediate release formulation.

Example Embodiment 29: The use of any one of example embodiments 22 to 28, wherein the medicament, when used in the reduction of body fat in a subject in need thereof, is administered to a region of the subject's body selected from the group consisting of buttocks, under the chin, underarm, under one or both eyes, cheek, brow, calf, back, thigh, ankle, and abdomen.

Example Embodiment 30: The use of any one of example embodiments 22 to 29, wherein the subject in need thereof has cellulite.

Example Embodiment 31: The use of example embodiment 30, wherein the medicament, when used in the reduction of body fat in the subject in need thereof, is administered to an area within or near the cellulite.

Example Embodiment 32: The use of any one of example embodiments 22 to 31, wherein the subject is not or has not been diagnosed with pulmonary arterial hypertension.

Example Embodiment 33: The use of any one of example embodiments 22 to 32, wherein the subject is a human.

Example Embodiment 34: Use of a PGI2 receptor agonist in the manufacture of a medicament for treating a subject in need thereof.

Example Embodiment 35: The use of example embodiment 34, wherein the subject is diagnosed with or at risk of obesity, diabetes mellitus, fatty liver disease, or a cardiovascular disease.

Example Embodiment 36: The use of example embodiment 34 or 35, wherein the agonist is selected from the group consisting of selexipag, FK-788, beraprost, iloprost, carbacyclin, cicaprost and treprostinil, or a pharmaceutically acceptable salt thereof.

Example Embodiment 37: The use of any one of example embodiments 22 to 34, wherein the subject is not otherwise in need of medication to treat obesity, diabetes mellitus, fatty liver disease, or a cardiovascular disease.

Example Embodiment 38: The use any one of example embodiments 34 to 37, wherein the subject is a human.

Example Embodiment 39: Use of a PGI2 receptor agonist in the manufacture of a medicament for activating lipolysis in an adipocyte.

Example Embodiment 40: The use of example embodiment 39, wherein the activating lipolysis results in a 50% to 300% increase in the production of glycerol by the adipocyte.

Example Embodiment 41: The use of example embodiment 39 or 40, wherein the agonist is selected from the group consisting of selexipag, FK-788, beraprost, iloprost, carbacyclin, cicaprost and treprostinil, or a pharmaceutically acceptable salt thereof.

Example Embodiment 42: The use of any one of example embodiments 39 to 41, wherein the adipocyte is a human adipocyte.

Example Embodiment 43: Use of a PGI2 receptor agonist in a method for reducing body fat in a subject in need thereof.

Example Embodiment 44: The use of example embodiment 43, wherein the agonist is selected from the group consisting of selexipag, FK-788, beraprost, iloprost, carbacyclin, cicaprost and treprostinil, or a pharmaceutically acceptable salt thereof.

Example Embodiment 45: The use of example embodiment 43, wherein the method comprises administering to the subject in need thereof a pharmaceutical composition comprising a PGI2 receptor agonist.

Example Embodiment 46: The use of example embodiment 45, wherein the agonist is selected from the group consisting of selexipag, FK-788, beraprost, iloprost, carbacyclin, cicaprost and treprostinil, or a pharmaceutically acceptable salt thereof.

Example Embodiment 47: The use of example embodiment 45 or 46, wherein the administering is via a subcutaneous or a transdermal injection.

Example Embodiment 48: The use of example embodiment 45 or 46, wherein the administering is via a dermal patch, a transdermal patch, or a subdermal depot.

Example Embodiment 49: The use of example embodiment 45 or 46, wherein the administering is topical.

Example Embodiment 50: The use of any one of example embodiments 45 to 49, wherein the pharmaceutical composition is a sustained release formulation.

Example Embodiment 51: The use of any one of example embodiments 45 to 49, wherein the pharmaceutical composition is an immediate release formulation.

Example Embodiment 52: The use of any one of example embodiments 45 to 51, wherein the administering to the subject comprises administering the agonist to a region of the body selected from the group consisting of buttocks, under the chin, underarm, under one or both eyes, cheek, brow, calf, back, thigh, ankle, and abdomen.

Example Embodiment 53: The use of any one of example embodiments 43 to 52, wherein the subject has cellulite.

Example Embodiment 54: The use of example embodiment 53, wherein the administering is to an area within or near the cellulite.

Example Embodiment 55: The use of and one of example embodiments 43 to 54, wherein the subject is not or has not been diagnosed with pulmonary arterial hypertension.

Example Embodiment 56: The use of any one of example embodiments 43 to 54, wherein the subject is a human.

Example Embodiment 57: Use of a PGI2 receptor agonist in a method for treating a subject in need thereof.

Example Embodiment 58: The use of example embodiment 57, wherein the subject is diagnosed with or at risk of obesity, diabetes mellitus, fatty liver disease, or a cardiovascular disease.

Example Embodiment 59: The use of example embodiment 57 or 58, wherein the agonist is selected from the group consisting of selexipag, FK-788, beraprost, iloprost, carbacyclin, cicaprost and treprostinil, or a pharmaceutically acceptable salt thereof.

Example Embodiment 60: The use of any one of example embodiments 43 to 57, wherein the subject is not otherwise in need of medication to treat obesity, diabetes mellitus, fatty liver disease, or a cardiovascular disease.

Example Embodiment 61: The use of any one of example embodiments 57 to 60, wherein the subject is a human.

Example Embodiment 62: Use of a PGI2 receptor agonist in a method for activating lipolysis in an adipocyte.

Example Embodiment 63: The use of example embodiment 62, wherein the activating lipolysis results in a 50% to 300% increase in the production of glycerol by the adipocyte.

Example Embodiment 64: The use of example embodiment 62 or 63, wherein the agonist is selected from the group consisting of selexipag, FK-788, beraprost, iloprost, carbacyclin, cicaprost and treprostinil, or a pharmaceutically acceptable salt thereof.

Example Embodiment 65: The use of any one of example embodiments 62 to 64, wherein the adipocyte is a human adipocyte.

Example Embodiment 66: A PGI2 receptor agonist for use in in a method for reducing body fat in a subject in need thereof.

Example Embodiment 67: The use of example embodiment 66, wherein the agonist is selected from the group consisting of selexipag, FK-788, beraprost, iloprost, carbacyclin, cicaprost and treprostinil, or a pharmaceutically acceptable salt thereof.

Example Embodiment 68: The use of example embodiment 66, wherein the method comprises administering to the subject in need thereof a pharmaceutical composition comprising a PGI2 receptor agonist.

Example Embodiment 69: The use of example embodiment 68, wherein the agonist is selected from the group consisting of selexipag, FK-788, beraprost, iloprost, carbacyclin, cicaprost and treprostinil, or a pharmaceutically acceptable salt thereof.

Example Embodiment 70: The use of example embodiment 68 or 69, wherein the administering is via a subcutaneous or a transdermal injection.

Example Embodiment 71: The use of example embodiment 68 or 69, wherein the administering is via a dermal patch, a transdermal patch, or a subdermal depot.

Example Embodiment 72: The use of example embodiment 68 or 69, wherein the administering is topical.

Example Embodiment 73: The use of any one of example embodiments 68 to 72, wherein the pharmaceutical composition is a sustained release formulation.

Example Embodiment 74: The use of any one of example embodiments 68 to 72, wherein the pharmaceutical composition is an immediate release formulation.

Example Embodiment 75: The use of any one of example embodiments 68 to 74, wherein the administering to the subject comprises administering the agonist to a region of the body selected from the group consisting of buttocks, under the chin, underarm, under one or both eyes, cheek, brow, calf, back, thigh, ankle, and abdomen.

Example Embodiment 76: The use of any one of example embodiments 66 to 75, wherein the subject has cellulite.

Example Embodiment 77: The use of example embodiment 76, wherein the administering is to an area within or near the cellulite.

Example Embodiment 78: The use of any one of example embodiments 66 to 77, wherein the subject is not or has not been diagnosed with pulmonary arterial hypertension.

Example Embodiment 79: The use of any one of example embodiments 66 to 78, wherein the subject is a human.

Example Embodiment 80: A PGI2 receptor agonist for use in a method for treating a subject in need thereof.

Example Embodiment 81: The use of example embodiment 80, wherein the subject is diagnosed with or at risk of obesity, diabetes mellitus, fatty liver disease, or a cardiovascular disease.

Example Embodiment 82: The use of example embodiment 80 or 81, wherein the agonist is selected from the group consisting of selexipag, FK-788, beraprost, iloprost, carbacyclin, cicaprost and treprostinil, or a pharmaceutically acceptable salt thereof.

Example Embodiment 83: The use of any one of example embodiments 66 to 80, wherein the subject is not otherwise in need of medication to treat obesity, diabetes mellitus, fatty liver disease, or a cardiovascular disease.

Example Embodiment 84: The use of any one of example embodiments 80 to 83, wherein the subject is a human.

Example Embodiment 85: A PGI2 receptor agonist for use in a method for activating lipolysis in an adipocyte.

Example Embodiment 86: The use of example embodiment 85, wherein the activating lipolysis results in a 50% to 300% increase in the production of glycerol by the adipocyte.

Example Embodiment 87: The use of example embodiment 85 or 86, wherein the agonist is selected from the group consisting of selexipag, FK-788, beraprost, iloprost, carbacyclin, cicaprost and treprostinil, or a pharmaceutically acceptable salt thereof.

Example Embodiment 88: The use of any one of example embodiments 85 to 87, wherein the adipocyte is a human adipocyte.

Example Embodiment 89: A method of reducing fat in an subject substantially as described herein.

Example Embodiment 90: The method of example embodiment 89, wherein the subject is a human.

Example Embodiment 91: A method of reducing fat in an subject in need thereof substantially as described herein.

Example Embodiment 92: The method of example embodiment 91, wherein the subject is a human.

Example Embodiment 93: The use of a PGI2 receptor agonist substantially as described herein.

Example Embodiment 94: A PGI2 receptor agonist substantially as described herein.

### BRIEF DESCRIPTION OF DRAWINGS

FIG. 1 is a graph showing effects of varying doses of PGI₂ receptor agonists on lipolytic activity in human adipocytes. Each data point represents the mean±SEM (Standard Error of Mean) of 2 to 8 experiments.

FIG. 2 is a graph showing glycerol release caused by IP agonists (left graph; Iso: isoproterenol, Cica: Cicaprost, FK: FK-788) and beta agonists (Salm: Salmeterol, Tulo: Tulobuterol, BTA: BTA-243, Mira: Mirabegron). Cicaprost (Cica), and FK-788 increased glycerol release in fully differentiated human adipocytes, but not in mouse fat tissue.

### DETAILED DESCRIPTION

### I. Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 70% to 80% is stated, it is intended that 71%, 72%, 73%, 74%, 75%, 76%, 77%, 78%, and 79%, are also explicitly disclosed, as well as the range of values greater than or equal to 70% and the range of values less than or equal to 80%.

Unless indicated otherwise herein, the term “about” is intended to include values (e.g., weight percentages) proximate to the recited range that are equivalent (e.g. bioequivalent) in terms of the functionality of the individual ingredient (e.g. active ingredient or excipient), the composition, or the embodiment. Furthermore, as will be understood by a skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

The singular forms “a,” “an,” and “the” include plural referents unless the context clearly dictates otherwise. Thus, for example, reference to an “excipient” includes a single excipient as well as two or more of the same or different excipients, and the like.

As used herein, “adipocytes” refer to the cells that primarily compose adipose tissue, specializing in storing energy as fat.

As used herein, “body fat” refers to loose connective tissue known as “adipose tissue” and is composed of adipocytes. Its main role is to store energy in the form of fat, although it also cushions and insulates the body. Two types of adipose tissue exist: white adipose tissue (WAT) and brown adipose tissue (BAT). Body fat can be present throughout the body of an individual, for example, beneath the skin (subcutaneous fat; e.g. cellulite), around internal organs (visceral fat), in bone marrow (yellow bone marrow), in breast tissue (breast fat), around the waist (waist fat; e.g. “love handles”), under the chin (submental fat), thigh tissue (thigh fat), and other regions of the body as would be identifiable to a skilled person (e.g. HIV associated lipodystrophy, steatoblepharon, and others).

The amount of body fat in an individual can be determined and/or estimated by a variety of methods identifiable to a skilled person. For example, body fat percentage (mass of body fat divided by body mass) can be estimated by techniques known to a skilled person such as hydrostatic (underwater) weighing, whole-body air displacement plethysmography, near-infrared interactance, dual energy X-ray absorptiometry, body average density measurement (in conjunction with use of the Brozek or Siri formulas), bioelectrical impedance analysis, anthropometric methods (e.g. skinfold measurements, ultrasound measurements, and estimations based on the subject's body mass index), magnetic resonance imaging, computed tomography, and other methods identifiable to a skilled person. Additionally, though not a direct measurement of body fat amount, an individual's body mass index (BMI) can also be indicative of the amount of body fat in an individual. Additionally, visual inspection can also reveal accumulated body fat such as in cellulite which can also be used as part of a quantitative measurement of cellulite (see, for example, Smalls, L. K., et al., International Journal of Cosmetic Science 2005, 27 (5), 295-295). Additional methods for determining and/or estimating the amount of body fat will be identifiable to a skilled person.

As used herein, a “triglyceride” (triacylglycerol, TAG or triglyceride) is an ester derived from glycerol and three fatty acids. It is the main constituent of animal fats.

As used herein, “triglyceride lipase” refers to lipases that hydrolyze ester linkages of triglycerides.

As used herein, “lipolysis” refers to the hydrolysis of lipids.

As used herein, a “receptor agonist” (such as a “PGI₂ receptor agonist” or “IP agonist”) refers to a type of receptor ligand or drug that provokes or activates a biological or functional response itself upon binding to a receptor. Agonists mediate their effects by binding to the active site or to allosteric sites on receptors, or they may interact at unique binding sites not normally involved in the biological regulation of the receptors activity. Agonist activity may be reversible or irreversible depending on the longevity of the agonist-receptor complex, which, in turn, depends on the nature of agonist receptor binding.

The term “subject” as used herein refers to human or non-human animal. As used herein, the terms “subject,” “individual” or “patient” are used interchangeably and refer to a vertebrate, preferably a mammal. Mammals include, but are not limited to, humans. A subject is “in need of reducing body fat” (such as a “subject in need thereof” in the methods for reducing body fat described herein) if the individual desires, is advised, or otherwise requires a reduction in body fat either for therapeutic or for cosmetic reasons.

The term “reducing” as in “reducing body fat” as used herein refers to a lowering in the amount, mass, or volume of body fat. Such reduction can be measured and determined by measuring the amount of fat according to one or more of the methods described herein at an initial time point prior to the administering of the compounds described herein (e.g. IP agonists) and then measuring the amount of body fat at various time points (e.g. during the period of administering the compounds described herein as well after the administering has ceased). For example, a subject's body weight can be measured prior to beginning a treatment regimen with the compounds described herein and then measured during and after the treatment regimen. A decrease in body weight is indicative of a reduction in body fat. Similarly, skinfold measurements and/or other techniques (e.g. magnetic resonance imaging and/or computerized tomography) can be made or performed along with the weight measurements where a decrease in the parameters measured by those techniques (i.e. body fat percentage) is indicative of fat reduction. Additionally, the reduction of fat can be determined qualitatively such as by photographing the whole body, or portions of the body, at various time points before, during, and after a treatment regimen where the reduction in fat can be determined by visual inspection of the images (e.g. by seeing a visible reduction and in the size and/or volume of a particular fat deposit such as submental fat, waist fat, cellulite, and other forms of body fat amenable to visual inspection).

As used herein, the term “administering” refers to introduction of a substance (e.g. the IP agonists described herein) into a body of a subject and/or application of a substance onto the body of a subject by a particular route. Routes of administration would be identifiable to a skilled person and include, for example, oral administration, parenteral administration (e.g. subcutaneous injection, intramuscular injection, and intravenous injection), sublingual administration, buccal administration, rectal administration, ocular administration, otic administration, inhalation routes (e.g. inhaling a mist containing the substance though the mouth or nose), topical administration, transdermal administration (e.g. via transdermal patches), administration via an implant device, and others identifiable to a skilled person.

Administration can be “local” when the compound is administered to a particular localized region of the body and only that region near the site of administration is exposed to the compound (e.g. topical application or subcutaneous application to a particular region of the subject's body.)

Similarly, administration can be “systemic” when the compound is administered such that the compound is exposed throughout the subject's body and may be found in one or more regions distant from the site of administration (e.g. orally or intravenously administering the compound such that the compound will be distributed in the blood and throughout various tissues and/or body regions resulting in fat reduction at those tissues and/or regions).

As used herein, “effective amount” means the amount of the subject PGI₂ receptor agonist that will elicit the biological or medical response of a cell, tissue, system, animal or human that is being sought by the person administering the PGI₂ receptor agonist.

As used herein, “pharmaceutically acceptable excipient,” “pharmaceutically acceptable carrier” and the like as used herein refer to pharmaceutical excipients, e.g., pharmaceutically, physiologically, acceptable organic or inorganic carrier substances suitable for administration and which do not deleteriously react with the active agent. In some embodiments, a pharmaceutical composition can be sterilized and/or mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds disclosed herein.

### II. PGI₂ Receptor Agonists

Described herein are studies showing that PGI₂ receptor agonists increase lipolysis in and glycerol release by fully differentiated human adipocytes. Without wishing to be bound by theory, the inventors believe that such studies provide the basis for using prostacyclin analogs or prodrugs thereof to activate lipolysis and to treat subjects in need of fat reduction or amelioration of symptoms associated with obesity. In some embodiments, the PGI₂ receptor agonist is a prostacyclin analogue which when incubated with a mature adipocyte in vitro as described herein increases glycerol production in the mature adipocyte by at least about 1.5, 2, 2.5, 3, 3.5 or 4 times the level of glycerol production by a mature adipocyte not treated with a PGI₂ receptor agonist.

Cicaprost (2-[(2E)-2-[(3aS,4S,5R,6aS)-5-hydroxy-4-[(3S,4S)-3-hydroxy-4-methylnona-1,-6-diynyl]-3,3a,4,5,6,6a-hexahydro-1H-pentalen-2-ylidene]ethoxy]acetic acid):

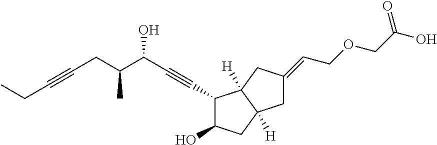 [SMILES: CCC#CC[C@H](C)[C@H](O)C#C[C@@H]1[C@H]2C/C(=C/COCC(=O)O)C[C@H]2C[C@H]1O]

has been shown to be chemically stable and highly pharmacologically potent (Hildebrand, 1992, Prostaglandins, 44:431-442). This prostacyclin analog is orally active as a vasodilator and an inhibitor of platelet aggregation. The synthesis of cicaprost has been described by Skuballa et al. in J. Med. Chem., 1986, 29, 313-316. In addition, analogues and prodrugs of cicaprost, which are also contemplated for use in the methods described herein (e.g. fat reduction methods), are described in U.S. Pat. Pub. No. 2014/0275266.

Beraprost (4-{(1R,2R,3aS,8bS)-2-Hydroxy-1-[(1E,3S)-3-hydroxy-4-methyl-1-octen-6-yn-1-yl]-2,3,3a,8b-tetrahydro-1H-benzo[b]cyclopenta[d]furan-5-yl}butanoic acid):

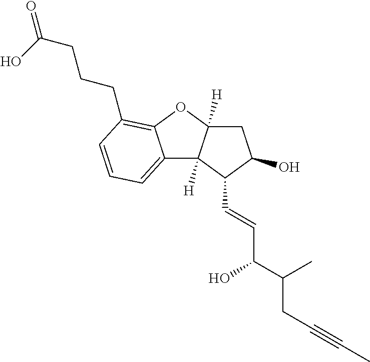 [SMILES: CC#CCC(C)[C@H](O)/C=C/[C@@H]1[C@H]2c3cccc(CCCC(=O)O)c3O[C@H]2C[C@H]1O]

is a synthetic benzoprostacyclin analogue of natural prostacyclin and is a PGI₂ receptor agonist having a vasodilating action and a platelet aggregation inhibition action. The sodium salt of beraprost (beraprost sodium) can be useful for improving ulcer, pain, and coldness associated with arteriosclerosis obliterans or thromboangiitis obliterans and is widely distributed in the ex-US market. Beraprost and related benzoprostacyclin analogues, which are also contemplated for use in the methods described herein (e.g. fat reduction methods), are described in U.S. Pat. No. 5,202,447 and Tetrahedron Lett. 31, 4493 (1990), and a synthesis of beraprost can be found in U.S. Pat. No. 8,779,170. Furthermore, as described in U.S. Pat. Nos. 7,345,181 and 4,474,802, several synthetic methods are known to produce benzoprostacyclin analogues. In some embodiments, the beraprost is beraprost sodium.

Iloprost (5-[(2E,3aS,4R,5R,6aS)-5-hydroxy-4-[(1E,3S)-3-hydroxy-4-methyloct-1-en-6-yn-1-yl]-octahydropentalen-2-ylidene]pentanoic acid):

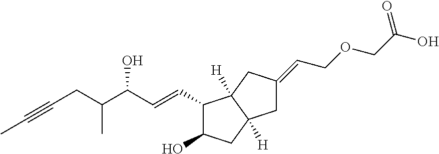 [SMILES: CC#CCC(C)[C@H](O)/C=C/[C@@H]1[C@H]2C/C(=C/COCC(=O)O)C[C@H]2C[C@H]1O]

is a synthetic prostacyclin analogue and is described in, for example, U.S. Pat. No. 5,663,203. A synthesis of iloprost can be found in Chandrasekhar, S., et al., Tetrahedron: Asymmetry 2012, 23 (5), 388-394 and Gais, H. J., et al., “Development of a Common Fully Stereocontrolled Access to the Medicinally Important and Promising Prostacyclin Analogues Iloprost, 3-Oxa-Iloprost and Cicaprost.” Chemistry—A European Journal 2006, 12 (21), 5610-5617.

FK-788 (2-[[(6R)-6-[di(phenyl)carbamoyloxymethyl]-6-hydroxy-7,8-dihydro-5H-naphthalen-1-yl]oxy]acetic acid):

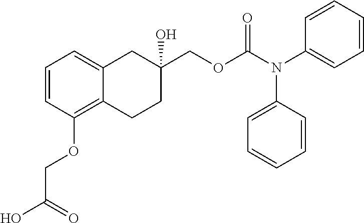 [SMILES: O=C(O)COc1cccc2c1CC[C@](O)(COC(=O)N(c1ccccc1)c1ccccc1)C2]

was developed and shown to be a highly potent and selective IP agonist. The synthesis of FK-788 can be found in Hattori et al., 2005, Bioorg. Med. Chem. Lett., 15:3091-3095.

Selexipag (2-(4-((5,6-diphenylpyrazin-2-yl)(isopropyl)amino) butoxy)-N-(methylsulfonyl)acetamide):

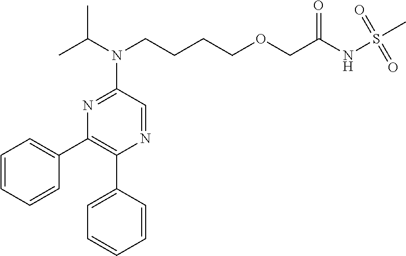 [SMILES: CC(C)N(CCCCOCC(=O)NS(C)(=O)=O)c1cnc(-c2ccccc2)c(-c2ccccc2)n1]

is an IP-selective agonist which is approved for treatment of pulmonary arterial hypertension. Syntheses of selexipag and functional analogues thereof, which are also contemplated for use in the methods described herein (e.g. fat reduction methods), are described in U.S. Pat. Nos. 7,205,302 and 8,791,122, in US Pat. App. Pub. No. US 2014/0155414, in PCT App. Pub. WO 2017/029594, and in Asaki, T., et al., “Structure-activity studies on diphenylpyrazine derivatives: a novel class of prostacyclin receptor agonists.” Bioorganic & medicinal chemistry 2007, 15 (21), 6692-6704.

Other epoprostenol analogs, which are also contemplated for use in the methods described herein (e.g. fat reduction methods), include 10,10-difluoro-13-dehydroprostacyclin, 11-desoxyprostacyclin, 13,14-dehydroprostaglandin I2, 13,14-dehydroprostaglandin I2 methyl ester, 13,14-didehydro-20-methylcarboprostacyclin, 13,14-dinor-inter-p-phenylene carbacyclin, 15-cyclopentyl-7-oxo-prostaglandin I2-ephedrine, 15-deoxy-(16-m-tolyl)-17,18,19,20-tetranorisocarbacyclin methylester, 15-deoxy-16-m-tolyl-17,18,19,20-tetranorisocarbacyclin, 15-fluoro-13,14-dehydrocarbacyclin, 15-ketoprostaglandin 12, 16-tolyl-17,18,19,20-tetranorisocarbacyclin, 17,20-dimethylisocarbacyclin, 19-(3-azidophenyl)-20-norisocarbacyclin, 2,2,10,10-tetrafluoro-13-dehydroprostacyclin, 20-methyl-13,14-didehydro-2,4-inter-3-phenylene prostaglandin I2, 3-oxa-9(O)-methano-delta(6,9)prostaglandin I(1), 3-oxacarbacyclin, 3-oxahomoisocarbacyclin, 4,5-didehydroisocarbacyclin, 5,6-dihydroprostacyclin, 5-hydroxyprostaglandin I, 5-methyleneisocarbacyclin, 5-nitroprostaglandin I1, 5-nitroprostaglandin I2, 6,9-thiaprostacyclin, 6a-carbaprostaglandin I3, 7-fluoroprostacyclin, 7-oxo-cyclopentyl-prostaglandin I2, 7-oxo-prostaglandin I2-ephedrine, 7-oxoprostaglandin I2, 7a-homo-2-norprostacyclin, 9-O-methanoprostaglandin I, AFP 03, AFP 06, AFP 07, APS 306, benzodioxane prostacyclin, bicyclo(4.3.0)non-2-ene homoisocarbacyclin, carbaprostacyclin, carboprostacyclin, CG 4303, Chinoin 7284, Chinoin 7384, ciprostene, CL 115999, dehydro-15-cyclohexylcarbaprostacyclin, dihomo-prostaglandin I(2), FCE 21258, HOE 892, homoisocarbacyclin, KP 10614, MM 706, naxaprostene, nileprost, nitriloprostaglandin 12, ONO 41483, OP 2507, OP 41483-α-cyclodextrin, piriprost, prostaglandin I2 11-methyl ether, prostaglandin I2 15-methyl ether, prostaglandin I2 methyl ester, prostaglandin I3, R 59274, SC 39902, SM 10902, SM 10906, taprostene, TEI 1324, TEI 3356, TEI 4343, TEI 9090, TFC 132, tilsuprost, treprostinil, TRY 200, TTC 909, TY 10957, TY 11223, U 56467, U 68215, and U 72382.

The present disclosure is directed to PGI₂ receptor agonist compounds as well as pharmaceutically acceptable salts thereof. Pharmaceutically acceptable salts can include salts of the active agonist compounds that are prepared with relatively nontoxic acids or bases, depending on the particular substituents found on the compounds described herein. When compounds disclosed herein contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds disclosed herein contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acid and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., Journal of Pharmaceutical Science, 1977, 66, 1-19). Certain specific compounds disclosed herein contain both basic and acidic functionalities that allow the compounds to be converted into either base or acid addition salts.

The compounds disclosed herein may exist as salts, such as with pharmaceutically acceptable acids. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid (see, e.g., Handbook of Pharmaceutical Salts, P. Heinrich Stahl & Camille G. Wermuth (Eds), Verlag; Helvetica Chimica Acta—Zürich, 2002, 329-345; and Berge et al., Journal of Pharmaceutical Science, 1977, 66:1-19). These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, embodiments disclosed herein provide compounds in a prodrug form. Prodrugs of the compounds described herein are those compounds that readily undergo chemical changes under physiological conditions to provide certain of the compounds disclosed herein. Additionally, prodrugs can be converted to certain compounds disclosed herein by chemical or biochemical methods in an ex vivo environment. For example, prodrugs can be slowly converted to certain compounds disclosed herein when placed in a transdermal patch reservoir with a suitable enzyme or chemical reagent. As an example, prodrugs of the IP agonist cicaprost are described in US patent application publication 2014/0275266.

Certain compounds disclosed herein can exist in unsolvated forms as well as solvated forms, including hydrated forms. In general, the solvated forms are equivalent to unsolvated forms and are encompassed within the scope of the present invention. Certain compounds disclosed herein may exist in multiple crystalline or amorphous forms. In general, all physical forms are equivalent for the uses contemplated by the present invention and are intended to be within the scope of the present invention.

Some compounds described herein have at least one asymmetric center in their structure. This asymmetric center can be present in an R or S configuration, said R and S notation is used in correspondence with the rules described in Pure Applied Chem. 1976, 45, 11-13.

Reference to a compound or compounds described herein is intended to encompass that compound in each of its possible isomeric forms and mixtures thereof unless the particular isomeric form is referred to specifically.

### III. Uses for PGI₂ Receptor Agonists

Lipolysis refers to a biochemical reaction in which a triglyceride is hydrolyzed into glycerol and free fatty acids (FFAs). The regulation of lipolysis plays a role in growth of adipocytes wherein an increase in lipolysis can be accompanied by a decrease or reduction in adipocyte size and/or mass of adipose tissue. Regulation of lipolysis can also affect the onset and/or progression of disorders such as obesity, insulin resistance, type 2 diabetes, dyslipidemia, hypertension and atherosclerosis.

As described in Example 1 below, experiments were performed in which human preadipocytes were differentiated in vitro to mature adipocytes. The mature adipocytes were exposed to varying doses of a PGI₂ receptor agonist and release of glycerol by these cells was measured as a direct indication of lipolysis activity in the cells. The results show that each of the PGI₂ receptor agonists increased lipolysis activity in adipocytes compared to adipocytes not treated with an agonist. The increase in glycerol production and secretion by the adipocytes ranged from about 1.5 times to about 3.5 times higher than glycerol production and release by untreated control adipocytes. Concentrations of PGI₂ receptor agonists effective in increasing lipolytic activity in this experiment ranged from about 0.001 μM to 100 μM. Accordingly, PGI₂ receptor agonists can be useful for reducing the size of adipocytes in a subject as well as reducing adipose tissue mass in a subject in need thereof.

Accordingly, in some embodiments, a method for increasing lipolysis activity is provided. The agonists increase lipolysis activity in an adipocyte as measured by production of glycerol wherein administration of the agonist to adipocytes in culture increases production of glycerol by the adipocytes by at least about 25%, 50%, 100%, 150%, 200%, 250% or 300%, or greater, with respect to the production of glycerol by adipocytes in the absence of administration of the PGI₂ receptor agonist.

Similarly, a method for reducing a mass of subcutaneous fat in a subject is provided comprising administering to the subject a PGI₂ receptor agonist as described in more detail below. Fat reduction can include reducing fat as measured by at least one of volume, size, mass, bulk, density, amount, and/or quantity. The presence, amount, or severity of excess fat can be assessed objectively, e.g., by magnetic resonance imaging (MRI), computed tomography, biopsy and histological analysis wherein tissue is sectioned and the reduction of fat mass is measured and compared to untreated areas, or skin calipers, or subjectively, e.g., by a clinician, a patient, or other observer, optionally with reference to a photonumeric, verbal, or descriptive scale or classification system, e.g., a five-step severity scale.

Local and/or total fat reduction can be greater than or equal to 75%, greater than or equal to 70%, greater than or equal to 60%, greater than or equal to 50%, greater than or equal to 40%, greater than or equal to 30%, greater than or equal to 25%, greater than or equal to 20%, greater than or equal to 15%, greater than or equal to 10%, or greater than or equal to 5%. Fat reduction can include reducing fat cell amount (for example, fat cell number), reducing fat cell volume, reducing fat cell maturation, and/or dedifferentiating a fat cell. The fat reduction can occur, for example, over a period of about 1 week, 2 weeks, 3 weeks, 4 weeks, 1 month, 2 months, 3 months, 4 months, 5 months, 6 months, 7 months, 8 months, 9 months, 10 months, 11 months, 1 year or 2 years.

Moreover, the increased lipolysis activity imparted by PGI₂ receptor agonists can be useful for the reduction of symptoms associated with various disorders including but not limited to diabetes mellitus, fatty liver disease, reperfusion injury, or a cardiovascular disease.

In some embodiments, a PGI₂ receptor agonist is used for reducing body fat in a subject in need thereof, wherein the agonist is regionally administered to a subject in need thereof. Regional administration can refer to administration to a localized region of the body and includes but is not limited to administration to the submental area (including the area under the chin), near the eye or eyebrow, the upper or lower arm, the buttocks, the inner thigh, the outer thigh, the medial region of the knees, the abdominal area, the lower back, the upper back, and/or the hip. In some embodiments, the subject has not been or is not diagnosed with pulmonary arterial hypertension. In other embodiments, the subject has not been or is not diagnosed with insulin resistance and/or diabetes mellitus.

### III. Pharmaceutical Compositions and Modes of Administration

Embodiments of the compositions are formulated for administration by any suitable method known in the art, for example, as described in Remington: The Science And Practice Of Pharmacy (21st ed., Lippincott Williams & Wilkins). PGI₂ receptor agonists can be formulated for various types of delivery e.g., subcutaneous, subdermal, intra-adipocyte, topical, intramuscular injection, intralesional injections etc. by any means known in the art. Preferably, compositions are formulated for localized or regional administration. Such formulations can be in the form of a solution, powder, gel, emulsion, cream, vapor, ointment, lotion, transdermal system, tablet, etc. Compositions comprising a PGI₂ receptor agonist or salt thereof can further comprise a pharmaceutically acceptable excipient. Examples of pharmaceutical excipients include: buffers, diluents, lubricating agents, solubilizers, solvents, surfactants, penetration enhancers, polymers, dispersion agents, wetting agents, emulsifying and suspending agents, and preserving agents.

The formulations of the present disclosure may be designed for to be short-acting, long-acting, immediate release, extended release, controlled release, or sustained release. The compositions disclosed herein may additionally include components that provide sustained release. Such components include but are not limited to high molecular weight, anionic mucomimetic polymers, gelling polysaccharides, and finely-divided drug carrier substrates. Non-limiting examples of these components are discussed in greater detail in U.S. Pat. Nos. 6,113,943; 6,630,155; and 5,807,573 and are known to the person having ordinary skill in the art.

The instant compositions may also comprise, for example, micelles or liposomes, or some other encapsulated form, or may be administered in an extended release form to provide a prolonged storage and/or delivery effect. Therefore, the pharmaceutical formulations may be compressed into pellets or cylinders and implanted intramuscularly or subcutaneously as depot injections or as implants such as stents. Such implants may employ known inert materials such as silicones and biodegradable polymers.

A PGI₂ receptor agonist is administered according to the desired therapeutic effect. For use, e.g., to reduce localized subcutaneous adipose tissues, administration of a PGI₂ receptor agonist can be localized to a particular region of the body such as but not limited to the skin of the buttocks, under the chin, periorbital skin, the cheek, the back, the abdomen, or a region of the thigh or arm. In an alternative embodiment, the PGI₂ receptor agonist is formulated for administration to treat or reduce symptoms of a non-regional disorder associated with excess adipose tissue including but not limited to diabetes mellitus, fatty liver disease and cardiovascular disease.

In some embodiments, the PGI₂ receptor agonist or salt thereof is formulated into a solution. In other embodiments, the solution is aqueous. The term “aqueous” as used herein refers to a solution which is a homogenous mixture prepared by dissolving a solid or a liquid in water such that the molecules of the solute or dissolved substance are dispersed among those of water. Examples of pharmaceutically acceptable aqueous vehicles include, without limitation, saline, water, benzyl alcohol and acetic acid. Typically, pharmaceutically acceptable aqueous vehicles are sterile. Solutions comprising the PGI₂ receptor agonist or salt thereof are suitable for at least subcutaneous, transdermal, intradermal and/or subdermal injections.

The pharmaceutical formulation may be in a powder form suitable for reconstitution with an appropriate solution. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these. A unit dosage form for injection may be in ampoules or in multi-dose containers.

For transdermal and topical administration, the PGI₂ receptor agonist can be formulated to enhance penetration to and across the stratum corneum of the skin. Those of ordinary skill in the art will be familiar with, or can readily ascertain the identity of, excipients and additives, which will facilitate drug delivery across skin. For review in this respect, reference may be made to “Novel Drug Delivery Systems”, Chien, ed (Marcel Dekker, 1992), the disclosure of which is incorporated herein by this reference to illustrate the state of knowledge in the art concerning drug delivery to and across the stratum corneum of the skin. Alternatively, the agonist can be formulated in a cream with an oil-in-water cream base. Topical formulation may further include, for example, antioxidants (e g, vitamin E); buffering agents; lubricants (e.g., synthetic or natural beeswax); sunscreens (e.g., para-aminobenzoic acid); and other cosmetic agents (e.g., coloring agents, fragrances, oils, essential oils, moisturizers or drying agents).

Other means of topical administration include delivery by electroporation, iontophoresis, phonophoresis, sonophoresis, and microneedle or needle-free injection for example using the systems sold under the trademarks POWDERJECT™, and BIOJECT™.

A PGI₂ receptor agonist may be administered via a transdermal patch. Transdermal patches may be shaped in a variety of sizes and forms suitable for treatment of specific body parts and condition. Methods of delivering a composition or compositions via a transdermal patch are known in the art. For example, patches and methods of patch delivery are described in U.S. Pat. Nos. 6,974,588, 6,564,093, 6,312,716, 6,440,454, 6,267,983, 6,239,180, and 6,103,275. Also contemplated is a transdermal patch employing iontophoresis, wherein an electric current is applied to enhance flux of ionized substances through membranes. In some embodiments, the patch comprises dissolvable microneedles. The pharmaceutical compositions herein can be packaged to produce a “reservoir type” transdermal patch with or without a rate-limiting patch membrane. Alternatively, the compositions herein can be formulated into a “matrix-type” transdermal patch. Drug Delivery Systems Characteristics and Biomedical Application, R. L Juliano, ed., Oxford University Press. N.Y. (1980); and Controlled Drug Delivery, Vol. I Basic Concepts, Stephen D. Bruck (1983) describe the theory and application of methods useful for transdermal delivery systems.

Also contemplated is administration of the PGI₂ receptor agonist using microporation. The microporation technique is ablation of the stratum corneum in a specific region of the skin using a pulsed laser light of wavelength, pulse length, pulse energy, pulse number, and pulse repetition rate sufficient to ablate the stratum corneum without significantly damaging the underlying epidermis. The agonist composition is then applied to the region of ablation.

Sonophoresis or phonophoresis is another microporation technique that uses ultrasound energy. Ultrasound is a sound wave possessing frequencies above 20 KHz. Ultrasound can be applied either continuously or pulsed, and applied at various frequency and intensity ranges (Nanda et al., Current Drug Delivery, 3:233 (2006)).

Another microporation technique involves the use of a microneedle array. The array of microneedles when applied to a skin region on a subject pierce the stratum corneum and do not penetrate to a depth that significantly stimulates nerves or punctures capillaries. The patient, thus, feels no or minimal discomfort or pain upon application of the microneedle array for generation of micropores through which the agonist is delivered.

Electroporation is another technique for creating micropores in the skin. This approach uses the application of microsecond or millisecond long high-voltage electrical pulses to created transient, permeable pores within the stratum corneum.

In some embodiments, when the compounds described herein are part of a composition, the compounds are the only active ingredients which have a therapeutic effect (e.g. fat reduction by way of lipolysis such that would be of use in reducing body fat in an individual in need thereof). The term “active ingredient” as used herein refers to a component which is responsible for the therapeutic effect or effects (e.g. fat reduction by way of lipolysis) of the composition, whereas the other components of the composition (e.g. excipients, carriers, and diluents) are not responsible for the therapeutic effect of the composition, even if they have other functions in the composition which are necessary or desired as part of the formulation (such as lubrication, flavoring, pH control, emulsification, and other functions other than the therapeutic effect or effects of composition as described herein). In particular, in some embodiments, compositions described herein in which the compound or compounds (e.g. IP agonists) are the only active ingredient or ingredients are compositions in which there are no other components which would be considered to have a therapeutic effect or effects. In particular, in some embodiments, compositions described herein in which the compound or compounds (e.g. IP agonists) are the only active ingredient or ingredients are compositions in which there are no other components which would be considered to have a fat-reducing effect. In other embodiments, compositions described herein in which the compound or compounds (e.g. IP agonists) are the only active ingredient or ingredients are compositions in which there are no other components which would be considered to have lipolytic activity.

### V. Effective Dosages

Pharmaceutical compositions contemplated herein include compositions wherein the active ingredient is contained in an effective amount, i.e., in an amount effective to achieve its intended purpose. An example of an “effective amount” is an amount sufficient to contribute to the treatment, prevention, or reduction of a symptom or symptoms of a disease, which can be referred to as a “therapeutically effective amount.” A “reduction” of a symptom or symptoms (and grammatical equivalents of this phrase) means decreasing of the severity or frequency of the symptom(s), or elimination of the symptom(s). The actual amount effective for a particular application will depend, inter alia, on the condition being treated. For example, when administered in methods to reduce subcutaneous adipose tissue, such compositions will contain amounts of active ingredients effective to achieve the desired result (e.g. decreasing the mass of adipose tissue in a subject).

The dosage and frequency (single or multiple doses) of compounds administered can vary depending upon a variety of factors, including route of administration; size, age, sex, health, body weight, body mass index, and diet of the recipient, nature and extent of symptoms of the disease being treated (e.g., the disease responsive to an PGI₂ receptor agonist), presence of other diseases or other health-related problems, kind of concurrent treatment, and complications from any disease or treatment regimen. Other therapeutic regimens or agents can be used in conjunction with the methods and compounds disclosed herein.

The therapeutically effective amount can be administered according to a dosing frequency that is identifiable to a skilled person during a time period that is also identifiable to a skilled person. The term “dosing frequency” as used herein, refers to the number of times the compounds described herein are administered to a subject. Exemplary dosing frequencies include administering the effective amount at discrete times during a day such as, for example, once a day (QD), twice a day (BID), three times a day (TID), four times a day (QID), and others identifiable to a skilled person. Other exemplary dosing frequencies include continuous dosing, for example by intravenous infusion, use of a drug pump, use of a transdermal patch, or other methods of continuous dosing identifiable to a skilled person.

The therapeutically effective amount can be administered at a desired dosing frequency for a time period identifiable to a skilled person. For example, a therapeutically effective can be administered once or twice a day (or at another dosing frequency identifiable to a skilled person) for a set period of time (e.g. seven to fourteen days, two to four weeks, one to six months, or for another time period identifiable to a skilled person). As another example, a therapeutically effective amount can be administered once or twice a day (or at another dosing frequency identifiable to a skilled person) for a non-predetermined period of time. A skilled person can determine at various points during the period of time if the administration of the effective amount is to be continued (e.g., if a desired outcome such as a particular amount of fat loss has been achieved and administration of the effective amount is not required and/or desired anymore).

For any compound described herein or combination thereof, the therapeutically effective amounts can be initially determined from cell culture assays. Target concentrations will be those concentrations of active compound(s) that are capable of increasing lipolysis (e.g., increasing glycerol production) as measured, for example, using methods known in the art.

Therapeutically effective amounts for use in humans may be determined from animal models. For example, a dose for humans can be formulated to achieve a concentration that has been found to be effective in animals. The dosage in humans can be adjusted by monitoring fat reduction and adjusting the dosage upwards or downwards, as described above.

Dosages may be varied depending upon the requirements of the subject and the compound being employed. The dose administered to a subject, in the context of certain embodiments disclosed herein, should be sufficient to effect a beneficial therapeutic response in the subject over time. The size of the dose also will be determined by the existence, nature, and extent of any adverse side effects. Generally, treatment is initiated with smaller dosages, which are less than the optimum dose of the compound. Thereafter, the dosage is increased by small increments until the optimum effect under circumstances is reached.

Dosage amounts and intervals can be adjusted individually to provide levels of the administered compounds effective for the particular clinical indication being treated. This will provide a therapeutic regimen that is commensurate with the severity of the individual's disease state.

The actual amount of the compound to be administered in any given case will be determined by a physician or other skilled person taking into account the relevant circumstances, such as the amount of fat reduction, the age and weight of the patient, the patient's general physical condition, the cause of the condition, and the route of administration.

The actual effective amount of the active compounds described herein also depends on the specific compound, and on the amount of fat reduction desired. The selection of the appropriate dose is well within the knowledge of the skilled artisan upon a reading of the present disclosure and based on the general knowledge of the skilled artisan. For example, in some subjects a reduction in body fat percentage to be in a range of about 25-31% from a higher percentage in females, and to be a range of about 15-24% from a higher percentage in males, can be a desired goal. Further lowering can be desirable and can be discussed between the subject and their healthcare provider so as to reduce fat in a medically safe manner. The dosage amounts and treatment duration can then be selected based on the subject's goal and the healthcare providers recommendation based on the medical knowledge of the healthcare provider. As another example, the amount of fat reduction can be an amount that results in at least about a 5% drop in body weight. As a further example, the amount of fat reduction can be an amount to result in a visible change in fat deposits (for example a visual reduction of submental fat, cellulite, abdominal fat, or waist fat). The compound can be administered in an effective amount until a desired visible change is achieved.

Other therapeutically efficient amounts will be apparent to a skilled person upon a reading of the present disclosure. For example, a skilled person can determine the maximum safe dosage for healthy subjects based on the dosages used in animal studies by routine methods (see, e.g. Food and Drug Administration, “Guidance for industry: estimating the maximum safe starting dose in initial clinical trials for therapeutics in adult healthy volunteers.” Center for Drug Evaluation and Research (CDER) 2005), and then administer to subjects in need thereof various dosages below the maximum safe dosage by routine methods and experimentation until a dosage which results in a desirable effect (e.g. fat reduction) is reached. Exemplary non-limiting effective amounts can be, for example, about 0.01 mg/kg to about 1 mg/kg, where mg/kg is milligrams of compound per kg of body weight of the subject. Additional non-limiting effective amounts can be, for example, about 0.01 mg/kg to about 0.1 mg/kg, about 0.01 mg/kg to about 0.2 mg/kg, about 0.01 mg/kg to about 0.3 mg/kg, about 0.01 mg/kg to about 0.4 mg/kg, about 0.01 mg/kg to about 0.5 mg/kg, about 0.01 mg/kg to about 0.6 mg/kg, about 0.01 mg/kg to about 0.7 mg/kg, about 0.01 mg/kg to about 0.8 mg/kg, and about 0.01 mg/kg to about 0.9 mg/kg.

Still additional non-limiting therapeutically effective amounts can be, for example, about 0.01 mg/kg to about 0.05 mg/kg, about 0.05 mg/kg to about 0.1 mg/kg, about 0.1 mg/kg to about 0.15 mg/kg, about 0.15 mg/kg to about 0.2 mg/kg, about 0.2 mg/kg to about 0.25 mg/kg, about 0.25 mg/kg to about 0.3 mg/kg, about 0.3 mg/kg to about 0.35 mg/kg, about 0.35 mg/kg to about 0.4 mg/kg, about 0.4 mg/kg to about 0.45 mg/kg, about 0.45 mg/kg to about 0.5 mg/kg, about 0.5 mg/kg to about 0.55 mg/kg, about 0.55 mg/kg to about 0.6 mg/kg, about 0.6 mg/kg to about 0.65 mg/kg, about 0.65 mg/kg to about 0.7 mg/kg, about 0.7 mg/kg to about 0.75 mg/kg, about 0.75 mg/kg to about 0.8 mg/kg, about 0.8 mg/kg to about 0.85 mg/kg, about 0.85 mg/kg to about 0.9 mg/kg, about 0.9 mg/kg to about 0.95 mg/kg, and about 0.95 mg/kg to about 1 mg/kg.

Still additional non-limiting therapeutically effective amounts can be, for example, about 0.001 mg/kg to about 0.0015 mg/kg, about 0.0015 mg/kg to about 0.002 mg/kg, about 0.002 mg/kg to about 0.0025 mg/kg, about 0.0025 mg/kg to about 0.003 mg/kg, about 0.003 mg/kg to about 0.0035 mg/kg, about 0.0035 mg/kg to about 0.004 mg/kg, about 0.004 mg/kg to about 0.0045 mg/kg, about 0.0045 mg/kg to about 0.005 mg/kg, about 0.005 mg/kg to about 0.0055 mg/kg, about 0.0055 mg/kg to about 0.006 mg/kg, about 0.006 mg/kg to about 0.0065 mg/kg, about 0.0065 mg/kg to about 0.007 mg/kg, about 0.007 mg/kg to about 0.0075 mg/kg, about 0.0075 mg/kg to about 0.008 mg/kg, about 0.008 mg/kg to about 0.0085 mg/kg, about 0.0085 mg/kg to about 0.009 mg/kg, about 0.009 mg/kg to about 0.0095 mg/kg, and about 0.0095 mg/kg to about 0.01 mg/kg.

Still additional non-limiting therapeutically effective amounts can be, for example, about 1 mg/kg to about 1.5 mg/kg, about 1.5 mg/kg to about 2 mg/kg, about 2 mg/kg to about 2.5 mg/kg, about 2.5 mg/kg to about 3 mg/kg, about 3 mg/kg to about 3.5 mg/kg, about 3.5 mg/kg to about 4 mg/kg, about 4 mg/kg to about 4.5 mg/kg, about 4.5 mg/kg to about 5 mg/kg, about 5 mg/kg to about 5.5 mg/kg, about 5.5 mg/kg to about 6 mg/kg, about 6 mg/kg to about 6.5 mg/kg, about 6.5 mg/kg to about 7 mg/kg, about 7 mg/kg to about 7.5 mg/kg, about 7.5 mg/kg to about 8 mg/kg, about 8 mg/kg to about 8.5 mg/kg, about 8.5 mg/kg to about 9 mg/kg, about 9 mg/kg to about 9.5 mg/kg, and about 9.5 mg/kg to about 10 mg/kg.

The therapeutically efficient amount can be present in a formulation (e.g. for topical administration), for example, at between about 0.01 and about 5% (w/v). In some embodiments, the therapeutically effective amount in the formulation can be, for example, from about 0.01 to about 1%, about 0.01 to about 2%, about 0.01 to about 3%, and about 0.01 to about 4% (w/v). In other embodiments, the therapeutically effective amount in the formulation can be, for example, from about 0.01 to about 1%, about 1 to about 2%, about 2 to about 3%, about 3 to about 4%, about 4 to about 5% (w/v).

In other embodiments, the therapeutically effective amount in the formulation can be, for example, from about 0.01 to about 0.06%, about 0.06 to about 0.11%, about 0.11 to about 0.16%, about 0.16 to about 0.21%, about 0.21 to about 0.26%, about 0.26 to about 0.31%, about 0.31 to about 0.36%, about 0.36 to about 0.41%, about 0.41 to about 0.46%, about 0.46 to about 0.51%, about 0.51 to about 0.56%, about 0.56 to about 0.61%, about 0.61 to about 0.66%, about 0.66 to about 0.71%, about 0.71 to about 0.76%, about 0.76 to about 0.81%, about 0.81 to about 0.86%, about 0.86 to about 0.91%, about 0.91 to about 0.96%, about 0.96 to about 1.01%, about 1.01 to about 1.06%, about 1.06 to about 1.11%, about 1.11 to about 1.16%, about 1.16 to about 1.21%, about 1.21 to about 1.26%, about 1.26 to about 1.31%, about 1.31 to about 1.36%, about 1.36 to about 1.41%, about 1.41 to about 1.46%, about 1.46 to about 1.51%, about 1.51 to about 1.56%, about 1.56 to about 1.61%, about 1.61 to about 1.66%, about 1.66 to about 1.71%, about 1.71 to about 1.76%, about 1.76 to about 1.81%, about 1.81 to about 1.86%, about 1.86 to about 1.91%, about 1.91 to about 1.96%, about 1.96 to about 2.01%, about 2.01 to about 2.06%, about 2.06 to about 2.11%, about 2.11 to about 2.16%, about 2.16 to about 2.21%, about 2.21 to about 2.26%, about 2.26 to about 2.31%, about 2.31 to about 2.36%, about 2.36 to about 2.41%, about 2.41 to about 2.46%, about 2.46 to about 2.51%, about 2.51 to about 2.56%, about 2.56 to about 2.61%, about 2.61 to about 2.66%, about 2.66 to about 2.71%, about 2.71 to about 2.76%, about 2.76 to about 2.81%, about 2.81 to about 2.86%, about 2.86 to about 2.91%, about 2.91 to about 2.96%, about 2.96 to about 3.01%, about 3.01 to about 3.06%, about 3.06 to about 3.11%, about 3.11 to about 3.16%, about 3.16 to about 3.21%, about 3.21 to about 3.26%, about 3.26 to about 3.31%, about 3.31 to about 3.36%, about 3.36 to about 3.41%, about 3.41 to about 3.46%, about 3.46 to about 3.51%, about 3.51 to about 3.56%, about 3.56 to about 3.61%, about 3.61 to about 3.66%, about 3.66 to about 3.71%, about 3.71 to about 3.76%, about 3.76 to about 3.81%, about 3.81 to about 3.86%, about 3.86 to about 3.91%, about 3.91 to about 3.96%, about 3.96 to about 4.01%, about 4.01 to about 4.06%, about 4.06 to about 4.11%, about 4.11 to about 4.16%, about 4.16 to about 4.21%, about 4.21 to about 4.26%, about 4.26 to about 4.31%, about 4.31 to about 4.36%, about 4.36 to about 4.41%, about 4.41 to about 4.46%, about 4.46 to about 4.51%, about 4.51 to about 4.56%, about 4.56 to about 4.61%, about 4.61 to about 4.66%, about 4.66 to about 4.71%, about 4.71 to about 4.76%, about 4.76 to about 4.81%, about 4.81 to about 4.86%, about 4.86 to about 4.91%, about 4.91 to about 4.96%, and about 4.96 to about 5% (w/v).

Utilizing the teachings provided herein, an effective therapeutic treatment regimen can be planned that does not cause substantial toxicity and yet is entirely effective to treat the clinical symptoms demonstrated by the particular patient. This planning should involve the careful choice of active compound by considering factors such as compound potency, relative bioavailability, patient body weight, presence and severity of adverse side effects, preferred mode of administration, and the toxicity profile of the selected agent.

### EXAMPLES

The following examples are illustrative in nature and are in no way intended to be limiting.

### Example 1

### Effects of PGI₂ Receptor Agonists on Adipocyte Lipolytic Activity

In order to demonstrate the effects of a PGI₂ receptor agonist on lipolytic activity, human adipocytes were differentiated from preadipocytes using methods routine in the art. Two weeks after the initiation of differentiation, differentiated cells appeared rounded with large lipid droplets apparent in the cytoplasm, and were thereby considered mature adipocytes. At this time, the differentiation medium was washed out. Two days after washing out the differentiation agents, the mature adipocytes were treated with different concentrations of PGI₂ receptor agonist compounds cicaprost, beraprost, FK-788 and selexipag. Lipolytic activity was assessed by the measurement of glycerol released into the medium from triglyceride breakdown. In this experiment, lipolysis was measured using a human adipocyte lipolysis assay kit (ZenBio, Inc., Research Triangle Park, N.C., Cat #LIP-1-SPF).

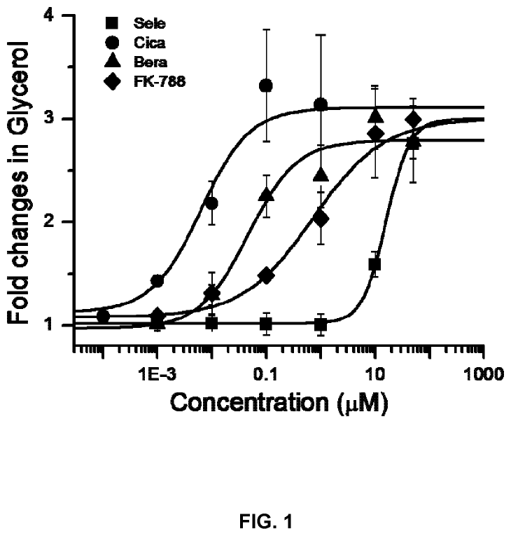

The data are provided in FIG. 1 and are expressed as fold induction over a buffer control (mature adipocytes treated with vehicle buffer in the absence of any agonist). Each data point on the graph in FIG. 1 represents the mean±SEM of 2 to 8 experiments.

The results show the varying activating effects of PGI₂ receptor agonists on lipolytic activity in human mature adipocytes resulting from a breakdown of intracellular triglycerides into free fatty acids and glycerol. The data show that in this particular assay system, the most potent agonist is cicaprost, showing an approximately 3-fold increase in glycerol production at a concentration of about 0.1 μM. Increases of about 1.5 to 3-fold by the various agonists was observed at concentrations ranging from about 0.001 μM to 10 μM.

### Example 2

### IP Agonists Possess Lipolytic Activity in Human Adipocytes Despite Low Lipolytic Activity in Rodents Models

The in vitro human adipocytes lipolysis results for cicaprost and FK-788 from Example 1 at the 1 μM concentration were compared to lipolysis induced by those compounds in ex vivo mouse adipocytes, which are commonly used to detect lipolytic activity. In addition, the lipolytic activity of several beta adrenergic agonists (isoproterenol (positive control), salmeterol, tulobuterol, BTA-243, and mirabegron) were also measured in human adipocytes (using the in vitro methods described in Example 1 at a concentration of 1 μM), and the results were also compared to the lipolysis induced by several of those compounds (isoproterenol, tulobuterol, BTA-243, and mirabegron) in ex vivo mouse adipocytes, where beta adrenergic agonists have been shown to have lipolytic activity.

For the ex vivo mouse adipocyte lipolysis measurements, after an acclimation period mice were sacrificed by cervical dislocation and adipose tissue (epididymal fat pad) was harvested from each animal. Adipose tissue was minced and incubated with 0.1% Collagenase A from Clostridium histolyticum (7 mL per gram of tissue) at 37° C. for 20 minutes, then rinsed in a Krebs buffer with bicarbonate, glucose, BSA and HEPES, and adipocytes were then isolated, counted. Approximately 300 to 500 thousand cells/mL in 1mL of lipolysis assay buffer with testing compounds (positive control: isoproterenol; PGI2 agonists: cicaprost and FK-788; beta agonists: tulobuterol, BTA-243, and mirabegron) was incubated for 3 hours at 37° C. in triplicate in basal conditions, with all compounds at 1 μM, except for the isoproterenol positive control which was at 10 nM. After the 3 hour incubation period, lipolysis was stopped by placing the tubes containing adipocytes on ice, enabling separation of cells from incubation medium over 10 minutes (no centrifugation step). Glycerol concentration was then measured from the incubation medium to evaluate lipolysis expressed as mg/million cells.

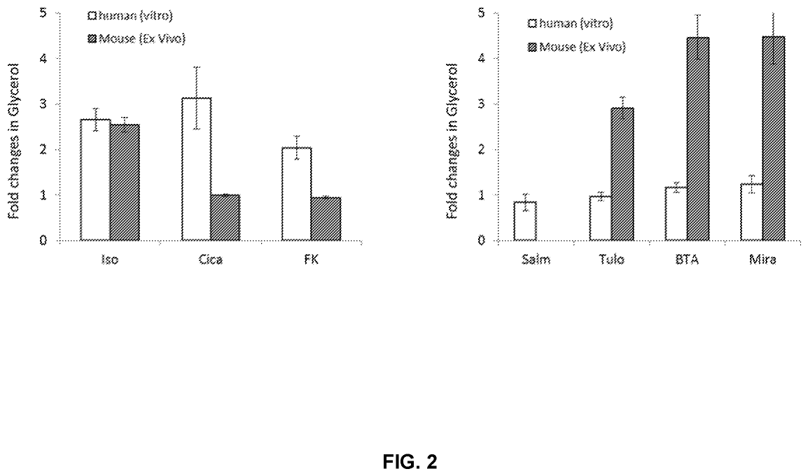

The data are provided in FIG. 2 and are expressed as fold induction over a buffer control (adipocytes treated with vehicle buffer in the absence of any agonist).

The results show that mouse adipocyte and mouse systems (a common method of screening compounds for lipolytic activity) would not have predicted the lipolytic activity shown by the IP agonists described herein. In FIG. 2, the IP agonists (left graph) show clear activity in human adipocytes but not in mouse. Similarly, beta agonists (right graph) show strong activity in mouse while little to no activity in humans. Thus, using a common mouse screening method, the IP agonists described herein would not have been expected to show lipolytic activity, especially when compared to beta agonists which do show greater lipolytic activity in the mouse screening model. However, as demonstrated in this Example and Example 1, the IP agonists do have significant lipolytic activity in human adipocytes. Furthermore, as also seen in FIG. 2, the lipolysis in human adipocytes induced by the IP agonists described herein is greater than the lipolysis in human adipocytes induced by the beta agonists.

Example 3

### In Vivo Mini-Pig Study

The following experiment describes a study in minipigs to test whether compounds according to the invention, including injectable FK-788, reduce dorsal skin fat.

Gottingen Minipigs are used. They are adult minipigs (approximately postnatal 3 months of age at study start). All animals are fed with Harlan Teklad Mini Swine Breeder Diet or comparable pig chow, appropriate for the species, ad libidum.

The back of the animals will be marked with forty-five (45) application sites. Each site will be 1 cm×1 cm square. There will be nine rows of application sites on each minipig. Each row will be composed of five squares, with a vehicle (V) injection site located on the third square in-between two replicates of drug application sites. Each of the two (2) replicates drug application sites will be located on either side of the vehicle injection sites. The first replicates will be located on the first and second squares, while the second replicates will be located on the fourth and fifth squares. While there will be no space in-between immediate adjacent drug application site, a 1 cm space will be separate the vehicle sites from the adjacent drug application sites to avoid cross contamination. Different rows will be applied different doses of FK-788 and will be separated from each other by a 1 cm space.

Animals will receive a single dose of obeticholic acid as positive control. Multiple SC doses of each of the five escalating doses of FK-788 (0.4 mg/cm2 to 0.0008 mg/cm2) and their respective vehicles will be administered daily over the course of seven day.

Body weights for the animals will be recorded prior to drug application and at the end of this study.

Three skin samples will be harvested from each row of the treatment sites for histological analysis: the first sample will contain the two left replicates, the second sample will contain the vehicle and adjacent block spaces and the third sample will contain the two right replicates. The head to tail orientation will be marked on the samples and the two set of replicates will be labeled “left” and “right”, respectively.

Histological analysis will be performed on these skin-fat samples to determine reduction of fat.

It is contemplated that the experiment described above will indicate that the test areas on animals treated with compounds according to the present invention, in this case FK-788, will exhibit lower subcutaneous (abdominal) fat mass, and/or lower total body mass compared to control areas and/or animals. These results may correlate in a dose-dependent manner within a range of FK-788 administration. As a result, these contemplated outcomes will show, in vivo, that compounds according to the present invention, including FK-788, can reduce fat.

### Example 4

### In Vivo Human Study

The following experiment describes a randomized, double-blind study in human subjects to test whether compounds according to the present disclosure, including topical FK-788, reduces fat in the body of individuals.

Multiple human subjects (both male and female), for example, with body mass indices of 30 or more but otherwise healthy, are entered into a randomized double-blind study. Either the left or the right arm is randomized to receive topical FK-788; the other arm receives vehicle only. FK-788 is supplied in a petrolatum-based ointment (the vehicle) at a final concentration of 0%, 0.003%, 0.03% or 0.3%. Ointment containers are unlabeled as to the presence or concentration of FK-788.

Each day, subjects apply a thin film of ointment to the skin over the respective triceps while wearing new, clean surgical gloves. Subjects are instructed to refrain from washing the treated area for at least 8 hours and are instructed to refrain from wearing tight clothing or occlusive dressings that will come into contact with the treated area.

Body fat calipers are used to measure fat in the triceps region bilaterally on a weekly basis. The study continues for 6 months. It is contemplated that the experiments described above will indicate that body areas, such as arms, treated with compounds according to the present invention, including FK-788, will exhibit lower fat compared to control areas, such as arms, treated with vehicle alone. These results may correlate in a dose-dependent manner within a range of FK-788 administration. As a result, this contemplated outcome will show, in vivo, that compounds according to the present invention, including FK-788, can reduce fat.

Throughout this specification reference is made to publications such as US and foreign patent applications, journal articles, book chapters, and others. All such publications are expressly incorporated by reference in their entirety, including supplemental/supporting information sections published with the corresponding references, for all purposes unless otherwise indicated.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. A method for reducing body fat in a subject in need thereof comprising administering to the subject in need thereof a pharmaceutical composition comprising a PGI2 receptor agonist.

2. The method of claim 1, wherein the agonist is selected from the group consisting of selexipag, FK-788, beraprost, iloprost, carbacyclin, cicaprost and treprostinil, or a pharmaceutically acceptable salt thereof.

3. The method of claim 1, wherein the administering is via a subcutaneous or a transdermal injection.

4. The method of claim 1, wherein the administering is via a dermal patch, a transdermal patch, or a subdermal depot.

5. The method of claim 1, wherein the administering is topical.

6. The method of claim 1, wherein the pharmaceutical composition is a sustained release formulation.

7. The method of claim 1, wherein the pharmaceutical composition is an immediate release formulation.

8. The method of claim 1, wherein the administering to the subject comprises administering the agonist to a region of the body selected from the group consisting of buttocks, under the chin, underarm, under one or both eyes, cheek, brow, calf, back, thigh, ankle, and abdomen.

9. The method of claim 1, wherein the subject has cellulite.

10. The method of claim 9, wherein the administering is to an area within or near the cellulite.

11. The method of claim 1, wherein the subject is not or has not been diagnosed with pulmonary arterial hypertension.

12. The method of claim 1, wherein the subject is a human.

13. A method for treating a subject in need thereof comprising administering a pharmaceutical composition comprising PGI2 receptor agonist.

14. The method of claim 13, wherein the subject is diagnosed with or at risk of obesity, diabetes mellitus, fatty liver disease, or a cardiovascular disease.

15. The method of claim 12, wherein the agonist is selected from the group consisting of selexipag, FK-788, beraprost, iloprost, carbacyclin, cicaprost and treprostinil, or a pharmaceutically acceptable salt thereof

16. The method of claim 1, wherein the subject is not otherwise in need of medication to treat obesity, diabetes mellitus, fatty liver disease, or a cardiovascular disease.

17. The method of claim 13, wherein the subject is a human.

18. A method for activating lipolysis in an adipocyte, comprising exposing the adipocyte to a PGI2 receptor agonist.

19. The method of claim 18, wherein the activating lipolysis results in a 50% to 300% increase in the production of glycerol by the adipocyte.

20. The method of claim 18, wherein the agonist is selected from the group consisting of selexipag, FK-788, beraprost, iloprost, carbacyclin, cicaprost and treprostinil, or a pharmaceutically acceptable salt thereof

21. The method of claim 18, wherein the adipocyte is a human adipocyte.

22.-94. (canceled)
